(19) United States
(12) Patent Application Publication
(10) Pub. No.: US 2012/0077753 A1
(43) Pub. Date: Mar. 29, 2012

(54) JNK INHIBITORS FOR USE IN TREATING SPINAL MUSCULAR ATROPHY

(76) Inventor: Laxman Gangwani, Evans, GA (US)

(21) Appl. No.: 13/321,029

(22) PCT Filed: Jun. 24, 2010
(86) PCT No.: PCT/US10/39779

§ 371 Date: Nov. 17, 2011

Related U.S. Application Data
(60) Provisional application No. 61/269,419, filed on Jun. 25, 2009.

(51) Int. Cl. A61K 31/7088; A61P 25/00; A61K 38/10; A61K 31/416; A61K 38/16

(52) U.S. Cl. 514/17.7; 514/406; 514/44 A

(57) ABSTRACT

The brain specific isoform (JNK3) of c-Jun NH2-terminal kinase (JNK) has been found to mediate the degeneration of spinal motor neurons caused by SMN deficiency in spinal muscular atrophy (SMA). Moreover, the ability of JNK inhibitors to reduce degeneration of neurons lacking SMN is also disclosed. The JNK signaling pathway can therefore mediate neurode-generation in SMA and represents a therapeutic target for treatment of SMA.

### CROSS REFERENCE TO RELATED APPLICATIONS

This application claims benefit of U.S. Provisional Application No. 61/269,419, filed Jun. 25, 2009, which is hereby incorporated herein by reference in its entirety.

### FIELD OF THE INVENTION

The invention is generally related to the use of c-Jun NH₂-terminal kinase (JNK) inhibitors for treating spinal muscular atrophy (SMA).

### BACKGROUND OF THE INVENTION

Spinal muscular atrophy (SMA) is the leading cause of infant death in the USA that results from an inherited genetic defect and is characterized by the degeneration of motor neurons in the anterior horn of the spinal cord (Markowitz, J A, et al. J Obstet Gynecol Neonatal Nurs. 2004 33(1):12-20). The clinical spectrum of SMA ranges from early infant death to normal adult life with only mild weakness. These patients often require comprehensive medical care involving multiple disciplines, including pediatric pulmonology, pediatric neurology, pediatric orthopedic surgery, pediatric critical care, and physical medicine and rehabilitation; and physical therapy, occupational therapy, respiratory therapy, and clinical nutrition.

SMA is caused by mutation of the Survival Motor Neurons (SMN) gene that results in low level expression of the full-length SMN protein (Lefebvre, S. et al. Cell. 1995 80(1):155-65; Lefebvre, S. et al. 1997 16(3):265-9). This genetic locus includes two copies of the SMN gene, SMN1 (telomeric) and SMN2 (centromeric) located in an inverted repeat on chromosome 5q13 (Lefebvre, S. et al. Cell. 1995 80(1):155-65). In 5q-linked SMA patients, the SMN1 gene is deleted or mutated and the SMN2 gene expresses transcripts that undergo alternative splicing due to a translationally silent nucleotide difference (C to T, codon 280) in exon 7 (Lorson, C L, et al. Proc Natl Acad Sci USA. 1999 96(10:6307-11). Alternative splicing of transcripts from the SMN2 gene causes skipping of exon 7 and predominant expression of a truncated SMNΔexon7 protein (Larson, C L, et al. Proc Natl Acad Sci USA. 1999 96(10:6307-11) that does not interact with many of the components of the SMN complex (Gangwani L, et al. Nat Cell Biol. 2001 3(4):376-83; Gubitz, A K, et al. 2004 296(1):51-6). This loss of expression of full-length SMN protein is a major cause of SMA (Oprea G E, et al. Science. 2008 Apr. 25; 320(5875):524-7).

It is established that the severity of SMA negatively correlates with the levels of full-length SMN protein, which is primarily influenced by number of SMN2 copies in SMA patients (Lefebvre, S. et al. Cell. 1995 80(1):155-65; Lefebvre, S. et al. 1997 16(3):265-9). However, the severity of SMA may also be influenced by the actions of other modifier genes.

The primary feature of SMA is muscle weakness, accompanied by atrophy of muscle. This is the result of denervation, or loss of the signal to contract, that is transmitted from the spinal cord. This is normally transmitted from motor neurons in the spinal cord to muscle via the motor neuron's axon, but either the motor neuron with its axon, or the axon itself, is lost in all forms of SMA.

Presently, treatment for SMA involves prevention and management of the secondary effect of chronic motor unit loss. There are currently no methods of treating or preventing the underlying motor neuron degeneration.

Thus, it is an object of the invention to provide improved compositions and methods for treating SMA.

### SUMMARY OF THE INVENTION

The brain specific isoform of c-Jun NH₂-terminal kinase (JNK) has been found to mediate the degeneration of spinal motor neurons caused by SMN deficiency in spinal muscular atrophy (SMA). JNK inhibitors have been found to reduce degeneration of neurons lacking SMN. One embodiment provides a method of inhibiting or reducing degeneration of neurons with reduced levels of SMN by contacting the one or more neurons with a JNK inhibitor. A method of treating one or more symptoms of SMA in a subject is also provided. The method includes administering to the subject one or more JNK inhibitors in an amount effective to reduce or inhibit neuronal degeneration.

### BRIEF DESCRIPTION OF THE DRAWINGS

FIG. 1 is a bar graph showing activation of Akt1 (first set of bars), Akt2 (second set of bars), and Akt3 (third set of bars) in primary neurons transfected with scrambled siRNA (control, black bars) or ZPR1 specific siRNA (siRNA-Zpr1, open bars).

FIG. 2 is a bar graph showing activation of JNK1 (first set of bars), JNK2 (second set of bars), and JNK3 (third set of bars) in primary neurons transfected with scrambled siRNA (control, black bars) or ZPR1 specific siRNA (siRNA-Zpr1, open bars).

### DETAILED DESCRIPTION OF THE INVENTION

### I. Definitions

The term “direct inhibitor” of a kinase refers to an inhibitor which interacts with the kinase or binding partner thereof or with a nucleic acid encoding the kinase.

The term “indirect inhibitor” of a kinase refers to an inhibitor which interacts upstream or downstream of the kinase in the regulatory pathway and which does not interacts with the kinase or binding partner thereof or with a nucleic acid encoding the kinase. Thus, for example, an indirect inhibitor of JNK can be an inhibitor of MEKK1.

The term “JNK pathway” refers to a signal transduction pathway in which at least one c-Jun NH₂-terminal kinase (JNK) enzyme is involved.

The term “subject” means any individual who is the target of administration. The subject can be a vertebrate, for example, a mammal. Thus, the subject can be a human. The term does not denote a particular age or sex. Thus, adult and newborn subjects, as well as fetuses, whether male or female, are intended to be covered.

The term “therapeutically effective” means that the amount of the composition used is of sufficient quantity to ameliorate one or more causes or symptoms of a disease or disorder. Such amelioration only requires a reduction or alteration, not necessarily elimination.

The terms “inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “peptide” refers to a natural or synthetic molecule having two or more amino acids linked by the carboxyl group of one amino acid to the alpha amino group of another. The peptide is not limited by length; thus “peptide” can include polypeptides and proteins.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “nucleic acid” may be used to refer to a natural or synthetic molecule having a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “small molecule” JNK inhibitor refers to small organic compounds, inorganic compounds, or any combination thereof that inhibits or reduces JNK activity; this term may include monomers or primary metabolites, secondary metabolites, a biological amine, a steroid, or synthetic or natural, non-peptide biological molecule(s).

### II. Compositions

The brain specific isoform (JNK3) of c-Jun NH₂-terminal kinase (JNK) has been found to mediate the degeneration of spinal motor neurons caused by SMN deficiency in spinal muscular atrophy (SMA). Further, treatment with JNK inhibitors has been found to reduce degeneration of neurons lacking or having reduced expression levels of SMN. These data indicate that the JNK signaling pathway mediates the neurodegeneration in SMA and represents a therapeutic target for treatment of SMA. Thus, one embodiment provides a method for reducing or inhibiting neuronal degeneration in a subject by administering to the subject an effective amount of one or more JNK inhibitors to inhibit or reduce neuronal degeneration.

A. JNK Inhibitor

The JNK inhibitors useful inhibiting neuronal degeneration can be any compound, molecule, protein, or nucleic acid identified as inhibiting one or more activities of JNK. “Activities” of a protein include, for example, transcription; translation; intracellular translocation; phosphorylation by kinases; enzymatic activity, including activity as a kinase to phosphorylate other proteins; homophilic and heterophilic binding to other proteins; and ubiquitination.

JNK has three isoforms, JNK1, JNK2 and JNK3, with several splice-variants of each for a total of ten different kinases ranging in molecular mass from 46 to 57 kDa. Thus, in certain embodiments JNK1, JNK2, JNK3 or combinations thereof are inhibited. Preferably, at least JNK3 is inhibited by the JNK inhibitor. In certain embodiments, one or more JNK splice variants are inhibited. The inhibitor can be a direct inhibitor or an indirect inhibitor.

In certain embodiments, the JNK inhibitor can be a compound that blocks, reduces or decreases the activity of JNK or the activity of a protein regulating JNK. For example, the inhibitor can decrease the JNK protein level or decrease expression of a gene encoding JNK. In still other embodiments, the JNK inhibitor can decrease the bioavailability of JNK.

JNKs are members of the mitogen-activated protein (MAP) kinase group which are activated in response to cytokines, such as TNF, e.g., TNF-α and IL-1, and exposure to environmental stress, including ultraviolet light, heat shock, and osmotic stress. Substrates of the JNK protein kinase include the transcription factors ATF2, Elk-1, and c-Jun. JNK phosphorylates each of these transcription factors within the activation domain and increases transcriptional activity. For example, JNKs phosphorylate Ser63 and Ser73 in the amino-terminal domain of the transcription factor c-Jun which results in increased transcriptional activity. The activity of a kinase can be reduced by inhibiting or reducing the interaction between the kinase and a substrate of the kinase or by inhibiting phosphorylation of the substrate. Thus, the activity of JNK can be inhibited by a compound which interferes with the interaction between a JNK and c-Jun.

JNKs are activated by dual phosphorylation at Thr183 and Tyr185 within the motifs Thr-Glu-Tyr and Thr-Pro-Tyr, respectively, by MKK4 and MAP kinase kinases. Although JNK is located in both the cytoplasm and the nucleus of quiescent cells, activation of JNK is associated with accumulation of JNK in the nucleus. The JNK inhibitor can inhibit activation of JNK by inhibiting phosphorylation of JNK, such as by inhibiting the interaction between JNK and the kinase that phosphorylates it. In one embodiment, the disclosed JNK inhibitor is a compound that interferes with the interaction between JNK and MKK4.

The JNK inhibitor can be an agent that inhibits MKK4. The JNK inhibitor can be an agent that blocks the action of activated c-Jun or c-Jun substrates. For example, the JNK inhibitor can be an artificial or recombinant membrane permeable peptide that can dilute the effect of activated c-Jun.

The JNK inhibitor can be an agent that inhibits JNK interacting protein (JIP). For example, Chen T, et al. Biochem J. 2009 May 13; 420(2):283-94, which is incorporated by reference, discloses small-molecule inhibitors that disrupt the JIP-JNK interaction to provide an alternative approach for JNK inhibition.

1. Compounds

The JNK inhibitor can be a compound, such as a small molecule. For example, the JNK inhibitor can include the compound SP600125 (Anthra[1,9-cd]pyrazol-6(2H)-on; 1,9-Pyrazoloanthrone) (Calbiochem., La Jolla, Calif.). A representative JNK inhibitor includes a compound having the formula:

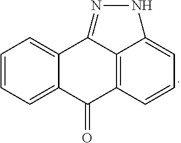

SP600125 is a potent and selective JNK-1, -2, and -3 inhibitor (Ki=0.19 μM). SP600125 is an ATP-competitive reversible inhibitor with >20-fold selectivity vs. a range of kinases and enzymes tested. In cells, SP600125 caused a dose-dependent inhibition of the phosphorylation of c-Jun. In animal studies, SP600125 inhibited bacterial lipopolysaccharide-induced expression of tumor necrosis factor-α and prevented anti-CD3-induced apoptosis of CD4+ CD8+ thymocytes (Bennett, B. L., et al. Proc Natl Acad Sci USA 2001 98:13681-86).

The JNK inhibitor can be a compound based on the 6,7-dihydro-5H-pyrrolo[1,2-a]imidazole scaffold (e.g., ER-181304). The JNK inhibitor can be SB203580.

The JNK inhibitor can be a selective inhibitor of JNK3. Selective inhibitors of JNKs are disclosed in International Patent Publication WO 2010/039647. Compounds 7-(5-7V-Phenylaminopentyl)-2H-anthra[1,9-cd]pyrazol-6-one; 7-(7-7V-Benzoylaminoheptyl)amino-2H-anthra[1,9-cd]pyrazol-6-one; and 7-(5-(p-Tolyloxy)pentyl)amino-2H-anthra[1,9-cd]pyrazol-6-one are selective inhibitors of JNK3.

The JNK inhibitor can be identified by the screening assays for the detection of inhibitors of protein kinase expression or activity disclosed in U.S. Patent Publication 2003/0023990, which is incorporated by reference in their entirety for the disclosure of these peptides. For example, the JNK inhibitor can be identified by a screening assays that involves incubating a cell that can express a JNK3 protein with a compound under conditions and for a time sufficient for the cell to express a JNK3 protein absent the compound; incubating a control cell under the same conditions and for the same time absent the compound; measuring JNK3 expression in the cell in the presence of the compound; measuring JNK3 expression in the control cell; and comparing the amount of JNK3 expression in the presence and absence of the compound, wherein a difference in the level of expression indicates that the compound modulates JNK3 expression.

2. Dominant Negative Proteins

Alternatively, the JNK inhibitor can be a dominant negative form of JNK. A catalytically inactive JNK-1 molecule functioning as a dominant inhibitor of the wild-type JNK-1 molecule is described, e.g., in International Patent Publication No. WO 1996/036642. This mutant was constructed by replacing the sites of activating Thr183 and Tyr185 phosphorylation with Ala and Phe, respectively.

3. Peptides

In some embodiments, the JNK inhibitor is a cell-permeable peptide that binds to JNK and inhibits JNK activity. No particular length is implied by the term “peptide.” In some embodiments, the JNK-inhibitor peptide is less than 280 amino acids in length, e.g., less than or equal to 150, 100, 75, 50, 35, or 25 amino acids in length. In some embodiments, the JNK inhibitor peptides bind JNK. In some embodiments the peptide inhibits the activation of at least one JNK activated transcription factor, e.g. c-Jun, ATF2 or Elk1.

Exemplary JNK peptide inhibitors are disclosed in U.S.S.N. 6,610,820 and U.S. Patent Publication 2009/0305968, which are incorporated by reference in their entirety for the disclosure of these peptides. For example, the JNK inhibitor include peptide having the amino acid sequence DTYRPKRPTT LNLFPQVPRS QDT (SEQ ID NO:1); EEPHKHRPTT LRLTTLGAQD S (SEQ ID NO:2); TDQSRPVQPF LNLTTPRKPR YTD (SEQ ID NO:3); or SDQAGLTTLR LTTPRHKHPE E (SEQ ID NO:4).

The JNK peptide inhibitor can be a JIP-1 polypeptide that binds JNK. Exemplary JIP-1 polypeptide inhibitors of JNK are disclosed in U.S. Patent Publications 2007/0003517 and 2002/0119135, which are incorporated by reference in their entirety for the disclosure of these peptides. For example, the JNK inhibitor include peptide having the amino acid sequence SGDTYRPKRPTTLNLFPQVPRSQDTLN (SEQ ID NO:12).

JNK-inhibitor peptides may be obtained or produced by methods well-known in the art, e.g. chemical synthesis, genetic engineering methods as discussed below. For example, a peptide corresponding to a portion of a JNK inhibitor peptide including a desired region or domain, or that mediates the desired activity in vitro, may be synthesized by use of a peptide synthesizer.

The JNK-inhibitor peptide can further constitute a fusion protein or otherwise have additional N-terminal, C-terminal, or intermediate amino acid sequences, e.g., linkers or tags. “Linker”, as used herein, is an amino acid sequences or insertion that can be used to connect or separate two distinct polypeptides or polypeptide fragments, wherein the linker does not otherwise contribute to the essential function of the composition. A polypeptide provided herein, can have an amino acid linker having, for example, the amino acids GLS, ALS, or LLA. A “tag”, as used herein, refers to a distinct amino acid sequence that can be used to detect or purify the provided polypeptide, wherein the tag does not otherwise contribute to the essential function of the composition. The provided polypeptide can further have deleted N-terminal, C-terminal or intermediate amino acids that do not contribute to the essential activity of the polypeptide.

The disclosed JNK inhibitors can be linked to an internalization sequence or a protein transduction domain to effectively enter the cell. Cell penetrating peptides include the TAT transactivation domain of the HIV virus, antennapedia, and transportan that can readily transport molecules and small peptides across the plasma membrane (Schwarze et al., Science. 1999 285(5433):1569-72; Derossi et al. J Biol. Chem. 1996 271(30):18188-93; Fuchs and Raines, Biochemistry. 2004 43(9):2438-44; and Yuan et al., Cancer Res. 2002 62(15):4186-90)). Nonaarginine has been described as one of the most efficient polyarginine based protein transduction domains, with maximal uptake of significantly greater than TAT or antennapeadia. Peptide mediated cytotoxicity has also been shown to be less with polyarginine-based internalization sequences. Polyarginine (R₉) mediated membrane transport is facilitated through heparan sulfate proteoglycan binding and endocytic packaging. Once internalized, heparan is degraded by heparanases, releasing R₉ which leaks into the cytoplasm (Deshayes et al., Cell Mol Life Sci. 2005 62(16):1839-49)). Studies have shown that derivatives of polyarginine can deliver a full length p53 protein to oral cancer cells, suppressing their growth and metastasis, defining polyarginine as a potent cell penetrating peptide (Takenobu et al., Mol Cancer Ther. 2002 1(12):1043-9)).

Additional cell penetrating peptides include, but are not limited to Penetratin, Antp-3A (Antp mutant), Buforin II, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol).

4. Nucleic Acids

The JNK inhibitor of the provided method can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of JNK or the genomic DNA of JNK or they can interact with the polypeptide JNK. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. In one embodiment the antisense molecules bind the target molecule with a dissociation constant (IQ) less than or equal to 10⁻⁶, 10⁻⁸, 10⁻¹⁰, or 10⁻¹² under physiological conditions. Methods for making antisense nucleic acids are well known in the art.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10¹² M under physiological conditions. It is preferred that the aptamers bind the target molecule with a K_{d} less than 10⁻⁶, 10⁻⁸, 10⁻¹⁰, or 10⁻¹² under physiological conditions. Aptamers can bind the target molecule with a very high degree of specificity. For example, aptamers have been isolated that have greater than a 10,000 fold difference in binding affinities between the target molecule and another molecule that differ at only a single position on the molecule. In one embodiment, the aptamer have a K_{d} with the target molecule at least 10, 100, 1000, 10,000, or 100,000 fold lower than the K_{d} with a background binding molecule. In one embodiment, the background molecule is a different polypeptide. Representative examples of how to make and use aptamers to bind a variety of different target molecules is well known in the art.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)). Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Mad. Sci. (USA) 92:2627-2631 (1995)).

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAs (shRNAs). Kits for the production of vectors including shRNA are available, such as, for example, Imgenex's GENESUPPRESSOR™ Construction Kits and Invitrogen's BLOCK-ITT™ inducible RNAi plasmid and lentivirus vectors. Disclosed herein are any shRNA designed as described above based on the sequences for the herein disclosed inflammatory mediators.

Thus, in some embodiments, the JNK inhibitor can be an inhibitory RNA such as an siRNA directed against expression of JNK, such as JNK1, JNK2 or JNK3. Some non-limiting examples of RNAi that inhibit JNK expression include: Jnk1/2 siRNA 5′-GAAUGUCCUACCUUCUCUA-3′ (SEQ ID NO:5); JNK 1 pool siRNA 5′-GGAAAGAACUGAUAUACAA-3′(SEQ ID NO:6) and 5′-GAAGCAAACGUGACAACAA-3′ (SEQ ID NO:7); JNK2 pool siRNA 5′-CCGUGAACUCGUCCUCUUAAA-3′ (SEQ ID NO:8) and 5′-GUGAUGGACUGGGAAGAAA-3′ (SEQ ID NO:9); JNK3 pool siRNA 5′-GAAAGAACUUAUCUACAA-3′ (SEQ ID NO:10) and 5-CCAGUAACAUUGUAGUCAA-3 (SEQ ID NO:11) (Bjorkblom, B., et al, J. Biol. Chem. 283: 19704-19713 (2004).

B. Pharmaceutically Acceptable Carriers

The disclosed compositions can be used therapeutically in combination with a pharmaceutically acceptable carrier. “Pharmaceutically acceptable” refers to a material that is not biologically or otherwise undesirable, i.e., the material may be administered to a subject, along with the JNK inhibitor, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more additional active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

### II. Methods

SMA is caused by a mutation of the Survival Motor Neurons 1 (SMN1) gene, which results in low level expression of the full-length SMN protein. A method for reducing or inhibiting degeneration of neurons having reduced or no expression of SMN includes contacting one or more neurons with a JNK inhibitor.

A method of treating SMA in a subject includes administering to the subject an effective amount of a JNK inhibitor to inhibit or reduce neuronal degradation relative to a control optionally in a pharmaceutically acceptable carrier. The JNK inhibitor can by any compound, molecule, peptide, or nucleic acid suitable for inhibiting one or more activities of JNK in vivo, inhibiting the expression of JNK, inhibiting the bioavailability of JNK, or a combination thereof. Suitable JNK inhibitors are described above.

A. Combination Therapies

The method can further involve administering to the subject a composition suitable for use in treating one or more symptoms of SMA. For example, the method can further involve administering one or more of classes of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin), steroids (e.g., Andranes (e.g., Testosterone), Cholestanes (e.g., Cholesterol), Cholic acids (e.g., Cholic acid), Corticosteroids (e.g., Dexamethasone), Estraenes (e.g., Estradiol), Pregnanes (e.g., Progesterone), narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenac Sodium, Diflorasone Diacetate; Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurbiprofen, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen, Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Morniflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone, Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolie Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tolmetin, Tolmetin Sodium, Triclonide, Triflumidate, Zidometacin, Zomepirac Sodium), or anti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine).

B. Administration

The disclosed compositions, including pharmaceutical composition, may be administered in a number of ways depending on whether local or systemic treatment is desired, and on the area to be treated. For example, the disclosed compositions can be administered parenterally, such as intravenously, intraperitoneally, intramuscularly, subcutaneously, intracavity, or transdermally. The compositions may be administered orally, ophthalmically, vaginally, rectally, intranasally, topically, or the like.

The disclosed compositions can be administered systemically or locally. For example, in some embodiments the disclosed compositions are administered orally or intravenously and cross the blood-brain barrier to reach motor neurons in the anterior horn of the spinal cord. In some embodiments, the disclosed compositions are administered to the cerebrospinal cavity.

Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

Transdermal formulations may also be prepared. These will typically be ointments, lotions, sprays, or patches, all of which can be prepared using standard technology. Transdermal formulations will require the inclusion of penetration enhancers. For example, the disclosed compositions can be delivered by a patch applied to the area of the spinal cord.

The disclosed compositions can be provided in sustained release composition. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

The exact amount of the compositions required can vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. Thus, effective dosages and schedules for administering the compositions may be determined empirically, and making such determinations is within the skill in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Guidance can be found in the literature for appropriate dosages for given classes of pharmaceutical products.

For example, a typical daily dosage of the JNK inhibitor used alone might range from about 1 μg/kg to up to 100 mg/kg of body weight or more per day, depending on the factors mentioned above. For example dosages can be about 0.01 to 5 mg/kg of the host body weight. For example dosages can be 0.3 mg/kg body weight, 1 mg/kg body weight, 3 mg/kg body weight 5 mg/kg body weight or 10 mg/kg body weight or within the range of 1-10 mg/kg body weight.

Dosage can vary, and can be administered in one or more dose administrations daily, for one or several days. An exemplary treatment regime entails administration twice per day, once per day, once per week, once every two weeks, once every three weeks, once every four weeks, once a month, once every 3 months or once every three to 6 months. Dosage can be a sustained release over several minutes, hours, or weeks from a single administration or application.

The JNK inhibitors may be administered prophylactically to patients or subjects who are at risk for SMA or who have been newly diagnosed with SMA.

### EXAMPLES

### Example 1

Materials and Methods

Neurons were isolated from the cerebellum of 7-day old mice and cultured in vitro for 6 days (Watson A, et al. J Neurosci. 1998 18(2):751-62). The cultured primary neurons were mock-transfected or transfected with control siRNA (Scramble) or Zpr1 specific siRNA (siRNA-Zpr1). Primary neurons were harvested 72 h post transfection and examined by immunoblot and immunofluorescence analysis, and phospho-MAPK Array analysis for Akt1, Akt2, Akt3, JNK1, JNK2, and JNK3.

Results

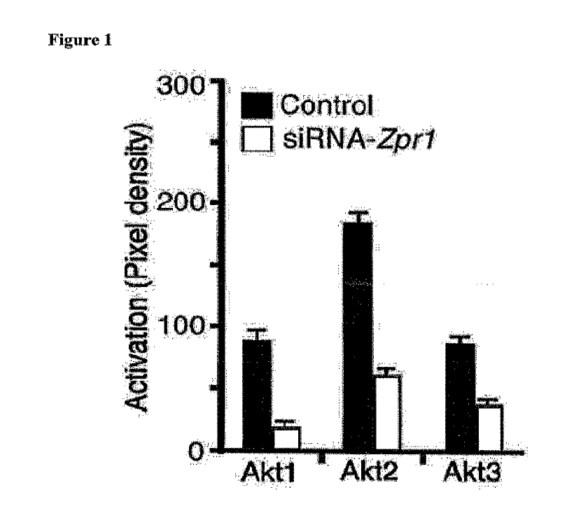

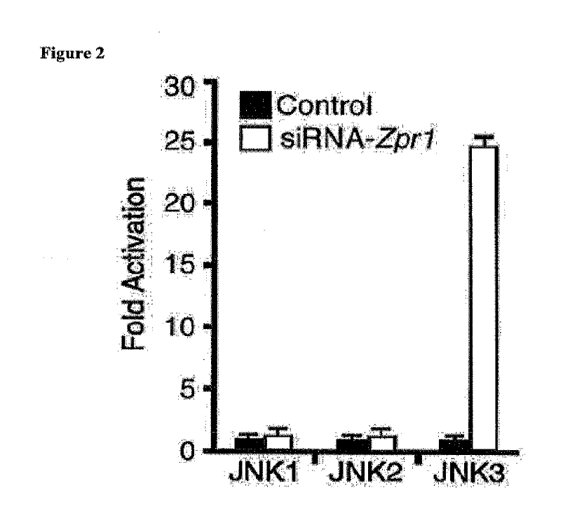

To understand the molecular mechanisms of neurodegeneration in SMA caused by ZPR1-deficiency, the effect of ZPR1-deficiency and SMN-deficiency on activation of MAP kinases and components of death signaling pathways was examined. The immunoblot analysis indicates that ZPR1-deficiency causes increase in the phosphorylation of c-Jun and activation of caspase-3. The phospho-MAPK analysis using Antibody Array (R&D Systems Inc.) shows that ZPR1-deficiency causes decrease in Akt activity (FIG. 1). It has been shown that the suppression of Akt-mediated survival is coupled with JNK-mediated death (Datta S R, et al. Genes Dev. 1999 13(22):2905-27; Suhara T, et al. Mol Cell Biol. 2002 22(2):680-91; Sunayama J, et al. J Cell Biol. 2005 170(2):295-304). These data indicate that the JNK is activated by the loss of ZPR1 expression. Interestingly, loss of ZPR1 in primary neurons caused a profound increase (25-fold) in the activity of JNK3, a neuronal specific isoform of JNK (FIG. 2) (Yang D D, et al. Nature. 1997 389(6653):865-70). These data indicate that loss of ZPR1 expression causes selective activation of JNK3 in primary neurons.

### Example 2

Materials and Methods

Primary cerebellar granule neurons from 7-day old mice transfected with scrambled siRNA (Control) and SMN specific siRNA (siRNA-Smn) were cultured for 72 h and stained with antibodies to Tubulin (neuron specific class III b-tubulin) and SMN. Stained neurons were examined by confocal microscopy. Neurons were also stained with antibodies to Tubulin and ZPR1.

Primary cerebellar granule neurons from 7-day old mice transfected with scrambled siRNA (Control) and ZPR1 specific siRNA (siRNA-Zpr1) were cultured for 72 h and stained with antibodies to Tubulin (neuron specific class III b-tubulin) and ZPR1. Neurons were also stained with antibodies to Tubulin and ZPR1.

Results

It has been shown that the reduced expression of SMN and reduced expression of ZPR1 causes neurodegeneration in mice (Doran B, et al. Proc Natl Acad Sci USA. 2006 103(19):7471-75; Frugier T, et al. Hum Mol. Genet. 2000 9(5):849-58; Jablonka S, et al. Hum Mol. Genet. 2000 9(3):341-46). To determine whether knockdown of SMN and ZPR1 causes neuron degeneration in cultured primary neurons, the effect of the Smn gene silencing was examined using RNA interference and immunofluorescence analysis. The examination of primary neurons treated with SMN specific siRNA (siRNA-Smn) shows that SMN-deficiency causes axon degeneration and mislocalization of ZPR1 protein. Knockdown of ZPR1 using RNAi causes axon degeneration, mislocalization of SMN and accumulation of SMN in the cytoplasm.

These results are consistent with previous findings and support the hypothesis that ZPR1 is required for nuclear accumulation of SMN (Gangwani L, et al. Mol Cell Biol. 2005 25(7):2744-56; Gangwani L, et al. Nat Cell Biol. 2001 3(4):376-83).

### Example 3

Materials and Methods

Primary cerebellar granule neurons transfected with scrambled siRNA (Control) and SMN specific siRNA (siRNA-Smn) were cultured for 72 h and stained with antibodies to Tubulin and phospho-c-Jun. Stained neurons were examined by confocal microscopy. Neurons were stained with antibodies to Tubulin and phosphoJNK.

Primary cerebellar granule neurons transfected with scrambled siRNA (Control) and ZPR1 specific siRNA (siRNA-Zpr1) were cultured for 72 h and stained with antibodies to Tubulin and phospho-c-Jun. Neurons were stained with antibodies to Tubulin and phosphoJNK.

Results

To determine whether SMN-deficiency causes JNK activation, the effect of SMN-deficiency on phosphorylation of c-Jun was first examined. The phosphorylation of c-Jun was not detected in neurons treated with scrambled siRNA (control). In contrast, neurons treated with SMN specific siRNA (siRNA-Smn) show robust increase in phosphorylation of c-Jun and nuclear accumulation. ZPR1-deficiency also causes phosphorylation and nuclear accumulation of c-Jun.

To determine whether the phosphorylation of c-Jun is caused by JNK activation, primary neurons lacking SMN or lacking ZPR1 were examined by staining with phosphospecific antibodies against activated JNK. SMN-deficiency results in phosphorylation (activation) and nuclear accumulation of JNK. Furthermore, ZPR1-deficiency also results in phosphorylation and nuclear accumulation of JNK.

These data show that both SMN-deficiency and ZPR1-deficiency cause JNK activation in neurons.

### Example 4

Materials and Methods

Primary ex-plant spinal cord neurons from 7-day old mice transfected with scrambled siRNA (Control) and SMN specific siRNA (siRNA-Smn) were cultured for 72 h and stained with antibodies to Tubulin (neuron specific class III b-tubulin) and SMN.

Primary neurons transfected with scrambled siRNA (Control) and ZPR1 specific siRNA (siRNA-Zpr1) were cultured for 72 hand stained with antibodies to Tubulin (neuron specific class III b-tubulin) and ZPR1.

Results

SMA is caused by degeneration of the spinal cord motor neurons therefore motor neurons represents relevant cell type. To understand the mechanism of degeneration of spinal motor neurons, the effect of ZPR1 and SMN deficiency was examine on degeneration culture mouse primary spinal cord neurons. The examination of primary neurons by immunofluorescence analysis shows that SMN-deficiency and ZPR1-deficiency causes axon degeneration and mislocalization of ZPR1 and SMN protein, respectively.

### Example 5

Materials and Methods

Primary neurons transfected with scrambled siRNA (Control) and SMN specific siRNA (siRNA-Smn) were cultured for 72 h and stained with antibodies to Tubulin and phospho-c-Jun. Stained neurons were examined by confocal microscopy. Neurons were also stained with antibodies to Tubulin and phospho-JNK. Phosphorylated JNK was observed in axons and in the nucleus. Axon swelling and degeneration was also observed.

Primary neurons transfected with scrambled siRNA (Control) and ZPR1 specific siRNA (siRNA-Zpr1) were cultured for 72 h and stained with antibodies to Tubulin and phospho-c-Jun. Stained neurons were examined by confocal microscopy. Neurons were also stained with antibodies to Tubulin and phospho-JNK. Phosphorylated JNK was observed in axons and in the nucleus. Axon swelling and degeneration was also observed.

Results

To determine whether SMN-deficiency also causes JNK activation in spinal cord neurons, phosphorylation of c-Jun was first examined. The phosphorylation of c-Jun was not detected in neurons treated with scam bled siRNA (control). In contrast, neurons treated with SMN specific siRNA show increase in phosphorylation of c-Jun.

To determine whether the phosphorylation of c-Jun is caused by activation of JNK, neurons stained with antibodies against activated JNK (phosphor-JNK) were examined. SMN-deficiency results in activation of JNK and causes axonal degeneration in spinal cord neurons. The knockdown of ZPR1 also resulted in phosphorylation of c-Jun and activation of JNK in spinal cord neurons.

These data show that deficiency of either SMN or ZPR1 result in activation of JNK and indicate that disruption of SMN complexes whether it is caused by reduced expression of SMN or ZPR1 result in activation of common target that may mediate neuron degeneration. Together, these data indicate that low levels of SMN cause activation of JNK and the JNK signaling pathway may mediate neuron degeneration in SMA.

### Example 6

Materials and Methods

Primary neurons transfected with scrambled siRNA (Control) and SMN specific siRNA (siRNA-Smn) were treated with DMSO or SP600125 after 36 h (post-transfection). Neurons were incubated for additional 60 h, fixed and stained with antibodies to Tubulin and phospho-JNK. Stained neurons were examined by confocal microscopy.

Primary neurons transfected with scrambled siRNA (Control) and SMN specific siRNA (siRNA-Smn) were treated with DMSO or SP600125 after 36 h (post-transfection). Neurons were incubated for additional 60 h, fixed and stained with antibodies to Tubulin and phospho-c-Jun. Stained neurons were examined by confocal microscopy.

Results

To determine whether JNK activity is required for neurodegeneration, the effect of JNK inhibitor (SP600125) was examine on degeneration of neurons caused by SMN-deficiency. Control experiments show that the treatment with (0.5 to 2 mm) of JNK inhibitor (SP600125) did not cause neuron degeneration. The knockdown of SMN neurons treated with solvent (DMSO) results in degeneration of neurons as expected. In contrast, neurons transfected with siRNA-Smn and treated with JNK inhibitor (SP600125) show marked reduction in degeneration in comparison to neurons treated with solvent. The comparison of neurons (control), treated with siRNA and treated with (siRNA+SP600125) indicates that inhibition of JNK may provide partial protection of neurons lacking SMN. To determine whether the neuroprotection is a result of JNK inhibition achieved by treatment of SP600125, the phosphorylation of c-Jun was examined. Treatment of neurons lacking SMN with SP600125 shows marked reduction in the phosphorylation of c-Jun and degeneration compared with neurons lacking SMN but untreated with JNK inhibitor. To determine whether activation of JNK is normal in neurons treated with siRNA and JNK inhibitor, phospho-JNK was examined because JNK is activated by phosphorylation. The presence of phospho-JNK in neurons treated with (siRNA+SP600125) indicates that JNK is activated by loss of SMN and it is the inhibition of JNK kinase activity by SP600125 contributes to neuroprotection. Together, these data suggest that JNK may mediate neurodegeneration caused by SMN-deficiency represent a potential therapeutic target for treatment of SMA.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

1. A method of inhibiting the degeneration of neurons with reduced Survival Motor Neurons (SMN) protein relative to healthy neurons, comprising contacting the neurons with an effective amount of a c-Jun NH₂-terminal kinase (JNK) inhibitor to inhibit or reduce neuronal degeneration relative to a control.

2. A method of treating one or more symptoms of spinal muscular atrophy (SMA) in a subject, comprising administering to the subject a composition comprising an effective amount of c-Jun NH₂-terminal kinase (JNK) inhibitor to inhibit or reduce neuronal degeneration relative to a control in a pharmaceutically acceptable excipient.

3. The method of claim 1, wherein the JNK inhibitor is 1,9-Pyrazoloanthrone (SP600125).

4. The method of claim 3, wherein the JNK inhibitor is administered to the subject at a daily dosage of 15 mg/kg.

5. The method of claim 1, wherein the JNK inhibitor comprises an RNAi molecule that reduces JNK expression.

6. The method of claim 5, wherein the RNAi molecule comprises the nucleic acid sequence SEQ ID NO:5, 6, 7, 8, 9, 10, or 11.

7. The method of claim 1, wherein the JNK inhibitor comprises a polypeptide that binds to JNK and inhibits JNK phosphorylation of c-Jun.

8. The method of claim 7, wherein the polypeptide comprises the amino acid sequence SEQ ID NO:1, 2, 3, or 4.

9. The method of claim 7, wherein the polypeptide comprises the amino acid sequence SEQ ID NO:12.


SEQUENCE LISTING (12 sequences)

SEQ ID NO: 1 - protein, 23 aa, Mus musculus
DTYRPKRPTTLNLFPQVPRSQDT

SEQ ID NO: 2 - protein, 21 aa, Mus musculus
EEPHKHRPTTLRLTTLGAQDS

SEQ ID NO: 3 - protein, 23 aa, Mus musculus
TDQSRPVQPFLNLTTPRKPRYTD

SEQ ID NO: 4 - protein, 21 aa, Mus musculus
SDQAGLTTLRLTTPRHKHPEE

SEQ ID NO: 5 - RNA, 19 nt, Mus musculus
gaauguccuaccuucucua

SEQ ID NO: 6 - RNA, 19 nt, Mus musculus
ggaaagaacugauauacaa

SEQ ID NO: 7 - RNA, 19 nt, Mus musculus
gaagcaaacgugacaacaa

SEQ ID NO: 8 - RNA, 21 nt, Mus musculus
ccgugaacucguccucuuaaa

SEQ ID NO: 9 - RNA, 19 nt, Mus musculus
gugauggacugggaagaaa

SEQ ID NO: 10 - RNA, 18 nt, Mus musculus
gaaagaacuuaucuacaa

SEQ ID NO: 11 - RNA, 19 nt, Mus musculus
ccaguaacauuguagucaa

SEQ ID NO: 12 - protein, 27 aa, Mus musculus
SGDTYRPKRPTTLNLFPQVPRSQDTLN